(19) United States
(12) Patent Application Publication
(10) Pub. No.: US 2007/0259014 A1
(43) Pub. Date: Nov. 8, 2007

(54) Compositions for and Methods for Treating Hiv

(75) Inventor: Jong Joseph Kim, North Wales, PA (US)

Correspondence Address: COZEN O'CONNOR, P.C., 1900 MARKET STREET, PHILADELPHIA, PA, 19103-3508, US

(73) Assignee: VGX PHARMACEUTICALS, INC., Blue Bell, PA (US)

(21) Appl. No.: 10/561,603

(22) PCT Filed: Jun. 21, 2004
(86) PCT No.: PCT/US04/19820

§ 371 Date: Mar. 29, 2007

Related U.S. Application Data
(60) Provisional application No. 60/480,500, filed on Jun. 20, 2003.
(60) Provisional application No. 60/480,393, filed on Jun. 20, 2003.

(51) Int. Cl. A61K 9/70; A61K 31/075; A61K 31/10; A61K 31/425; A61K 31/445; A61K 31/497; A61K 31/505; A61K 31/535; A61K 31/55; A61K 31/56; A61P 31/18; A61K 31/58; A61K 31/655; A61K 31/66; A61K 31/70; A61K 35/74; A61K 38/21; A61K 9/00

(52) U.S. Cl. 424/402; 424/425; 424/486; 424/780; 424/85.7; 514/120; 514/150; 514/172; 514/177; 514/179; 514/220; 514/230.5; 514/253.01; 514/256; 514/26; 514/274; 514/328; 514/365; 514/709; 514/716

(57) ABSTRACT

Pharmaceutical composition comprising compounds and/or compositions useful to inhibit HIV replication are disclosed. Methods of treating individuals infected with HIV are disclosed. Methods of preventing HIV infection in high risk individuals are disclosed.

### FIELD OF THE INVENTION

The present invention relates to pharmaceutical compositions which inhibit or prevent HIV replication, to methods of treating individuals who are infected with human immunodeficiency virus (HIV) infected, and to methods of preventing HIV infection in individuals who are exposed to HIV.

### BACKGROUND OF THE INVENTION

HIV is a lentivirus whose genome contains only about 9-11 kb of genetic material and less than 10 open reading frames. HIV possesses a collection of small, positive strand open reading frames which encode 1-2 exon genes whose protein products regulate various aspects of the virus' life cycle. Some of these genes are genetic transactivating factors which are necessary for virus replication in all permissive cell types.

The progression from HIV infection to AIDS is in large part determined by the effects of HIV on the cells that it infects, including CD4⁺ T lymphocytes and macrophages. Cell activation, differentiation and proliferation in turn regulate HIV infection and replication in T cells and macrophages. Gallo, R. C. et al. (1984) Science 224:500; Levy, J. A. et al., (1984) Science 225:840; Zack, J. A. et al. (1988) Science 240:1026; Griffin, G. E. et al., (1988) Nature 339:70; Valentin, A. et al. (1991) J. AIDS 4:751; Rich, E. A. et al., (1992) J. Clin. Invest. 89:176; and Schuitemaker, H. et al. (1992) J. Virol. 66:1354. Cell division per se may not be required since HIV and other lentiviruses can proliferate in nonproliferating, terminally differentiated macrophages and growth-arrested T lymphocytes. Rose, R. M. et al. (1986) Am. Rev. Respir. Dis. 143:850; Salahuddin, S. Z. et al. (1986) Blood 68:281; and Li, G. et al. (1993) J. Virol. 67:3969. HIV infection of myeloid cell lines can result in a more differentiated phenotype and increase the expression of factors such as NF-KB which are necessary for HIV replication. Roulston, A. et al. (1992) J. Exp. Med. 175:751; and Chantal Petit, A. J. et al. (1987)J. Clin. Invest. 79:1883.

Since the demonstration in 1987 that the small open reading frame within HIV-1 designated R encodes a 15 KD protein (Wong-Staal, F., et al., (1987) AIDS Res. Hum. Retroviruses 3:33-39), there has been a growing body of literature regarding the function of the viral protein R (Vpr). The ability of lentiviruses, including HIV, to replicate in nonproliferating cells, particularly in macrophages, is believed to be unique among retroviruses. It is significant that several lentiviruses contain a vpr-like gene. Myers, G. et al. (1992) AIDS Res. Hum. Retrovir. 8:373. The vpr open reading frame is conserved within all genomes of HIV-1 and HIV-2 and within all pathogenic isolates of simian immunodeficiency virus (SIV) genomes. The evolutionary requirement for economy in design is deemed to require that the presence of vpr in the HIV genome is related to a specific and non-dispensable function in the viral life cycle.

It has been reported that mutations in the vpr gene result in a decrease in the replication and cytopathogenicity of HIV-1, HIV-2, and SIV in primary CD4⁺ T lymphocytes and transformed T cell lines. See, e.g., Ogawa, K., et al., (1989) J. Virol. 63:4110-4114; Shibata, R., et al. (1990a) J. Med. Primatol. 19:217-225; Shibata, R., et al. (1990b) J. Virol. 64:742-747 and Westervelt, P. et al. (1992) J. Virol. 66:3925, although others have reported that mutated vpr gene had no effect on replication (Dedera, D., et al. (1989) Virol. 63:3205-3208). Importantly, HIV-2 mutated for vpr has been reported unable to infect primary monocyte/macrophages (Hattori, N., et al. (1990) Proc. Natl. Acad. Sci. USA 87:8080-8084). Further, viral replication in macrophages may be almost completely inhibited by antisense ribonucleotides targeting the vpr open reading frame. This, together with the induction of rhabdomyosarcoma cellular differentiation, are deemed to dictate a crucial function for Vpr in HIV pathogenesis.

The Vpr protein is the only HIV-1 regulatory gene product which has been shown to be incorporated into virions. This would normally suggest a structural role for Vpr, but since vpr deleted viruses are able to produce normal virions, this is deemed to be further evidence of a regulatory role for this molecule. The presence of Vpr in virions has been associated with increased replication kinetics in T lymphocytes, and with the ability of HIV to establish productive infection in monocytes and macrophages. The presence of Vpr protein in viral particles means an early function for Vpr during the infection process, following virus penetration and uncoating. This role is considered to involve Vpr interaction with cellular regulatory mechanisms resulting in an increase in cell permissiveness to sustain viral replication processes. See, e.g., Cohen, E. A., et al. 1990a J. Virol. 64:3097-3099; Yu, X. F., et al. (1990) J. Virol. 64:5688-5693; and, Yuan, X., et al., (1990) AIDS Res. Hum. Retroviruses 6:1265-1271.

U.S. Pat. No. 5,874,225, which is incorporated herein by reference, discloses several activities and characteristics of Vpr including its ability to inhibit cellular proliferation and its ability to associate with protein product encoded by the gag gene. Vpr action can involve the upregulation of cellular elements which enhance viral gene expression, or the downmodulation of cellular inhibitory pathways affecting such viral processes. Such cellular disregulation is consistent with the observation that Vpr is sufficient for the differentiation and cessation in cellular proliferation of rhabdomyosarcoma and osteosarcoma cell lines (Levy, D. N. et al. (1993) Cell 72:541). The ability of a virally associated protein such as Vpr to reinitiate an arrested developmental program is clearly based upon its interaction with other cellular proteins, and since Vpr protein originates within viral particles, it is considered that Vpr must, accordingly, play a role in establishing productive infection.

U.S. Pat. No. 5,780,238, which is incorporated herein by reference, describes the isolation of an approximately 41 KD Vpr cytosolic binding or interacting protein, which has been designated hereafter as Rip-1. As used herein, the term “Rip-1” is meant to refer to the human protein that has an apparent molecular weight of between 40-43 KD, that occurs in the cytoplasm of human cells, that binds to Vpr and that is transported from the cytoplasm to the nucleus when bound to Vpr, either alone or in association with a steroid receptor.

Rip-1 may be co-localized with the T-cell and B-cell transcription factor NfκB. Vpr and Rip-1 coelute in an immunoaffinity system, and can be specifically crosslinked to a 58 KD complex. Using peptide and antibody competition, the site of their interaction has been resolved to amino acids 38 to 60 on the Vpr amino acid sequence. Rip-1 has been detected in various cell lines. Rip-1 selectively translocates from the cytosol to the nucleus upon exposure of the cell to Vpr either in a soluble form, or through infection with wild type virus, but not in response to PMA, suggesting a coupling in their regulatory functions. Consequently, the present invention involves the discovery that Rip-1 may be partially responsible for mediating Vpr activity in the human host cell.

U.S. Pat. No. 5,639,598, which is incorporated herein by reference, refers to the discovery that HIV Vpr protein forms a complex with proteins, including Rip-1, in human cells that are in association with, i.e., as a part of or functionally combined with, one or more steroid receptors, especially the glucocorticoid receptor (GR). Inhibitory or antagonist compounds which bind to, or otherwise wholly or partially preclude the formation of a complex involving Vpr and steroid receptors, especially a GR-type receptor, or potentially other components, or one or more steroid receptors alone, prevent or interfere with HIV replication.

Rip-1 functions in association with one or more members of the steroid hormone receptor superfamily, and particularly, in association with one or more members of the glucocorticoid receptor (GR) family, and more particularly, in association with one or more members of the GR-type II receptor family. By “in association with” is meant that Rip-1 is a part of, forms a discrete complex with, or is functionally interactive or combined with, one or more of said steroid receptors. Thus, the Vpr, Rip-1, and steroid receptor or other component may be chemically and/or physically bound together to form a multi-part complex.

The cellular trafficking characteristics which have been observed for Rip-1 are consistent with Rip-1 functioning in association with, or even being a member of the steroid hormone receptor superfamily. The glucocorticoid and mineralocorticoid receptors are examples of members of this protein family which are known to translocate from the cytoplasm to the nucleus upon exposure to their ligand. Two types of glucocorticoid receptors have been described. Type I receptors are concentrated in the nucleus even when there is no ligand present. Type II receptors specifically concentrate in the cytoplasm in the absence of ligand, and only translocate to the nucleus in the presence of their appropriate stimulating hormone. The two types of glucocorticoid receptors have high affinity for their specific ligands, and are considered to function through the same transduction pathways. The main functional difference between these two classes of receptors is that the type II receptors are activated by their ligands in such a way that they only transactivate their target cellular protooncogenes in some, but not in all cells. Such cellular specificity is not observed in type I receptors. These observations are consistent with Rip-1 being functionally closely associated with, or actually being a GR-type II molecule.

Glucocorticoid receptors have a number of roles. Glucocorticoid receptors have been shown to act as powerful transactivators. Glucocorticoid receptors have also been shown to operate through the repression of gene expression for particular open reading frames. Glucocorticoid receptor mediated repression is attained by competition for the sites on the DNA molecule which would otherwise be bound by transactivators. An example of the latter is the specific bilateral relationship which has been described for glucocorticoid receptors and c-Jun. In this case, the glucocorticoid receptor represses c-Jun activity, and the opposite is also observed. The phorbol ester PMA has been reported to activate transcription of the AP-1/c-Jun promoter. In addition, glucocorticoids have been shown to counter lymphokine activity as observed by the inhibition of proliferation of a variety of cell lines. This mechanism is deemed to affect immunoregulatory mechanisms in areas such as T cell activation, which is in part mediated by the Jun/AP-1 activity, and its resulting lymphokines. The observation of a cessation in proliferation in different cell lines transfected with Vpr is considered explained by a glucocorticoid receptor mediated pathway, in which Rip-1, alone or in association with one or more steroid receptors or other components, or one or more steroid receptors, acts to bridge viral and cellular activities.

It is also important to note that the glucocorticoid receptors function as a part of a larger multimeric complex. These 330 KD protein clusters comprise a heat shock protein 90 dimer, a heat shock protein 56 unit, and sometimes by a heat shock protein 70 unit (HSP 70), in addition to the specific glucocorticoid receptor molecule; and Rip-1 has been observed in association with this HSP 70. The glucocorticoid receptor polypeptide itself is usually composed of three functional domains arranged in a linear configuration; a hormone binding domain, a DNA binding domain, and a third domain which has been shown to interact with additional cellular proteins, defining the trafficking characteristics of this gene product. It is contemplated that the complex comprising Rip-1, Vpr, and a steroid receptor or other components, may include as an example of the other components, the heat shock protein units described above.

Since Rip-1 in human cells appears to act in conjunction with a member of the steroid hormone receptor superfamily, especially the glucocorticoid receptor family, this may elucidate the manner in which the binding of Vpr to Rip-1 is involved in HIV replication and thus pathogenesis. Accordingly, interactively blocking Rip-1 or a complex including Rip-1 effectively inactivates Vpr and prevents it from converting cells to better HIV replication hosts. The identification of compounds which can inhibit the effects of Vpr and thereby inhibit HIV replication in HIV infected cells is based on the discovery that many of the actions of Vpr are analogous to those of a glucocorticoid The mechanism of action of Vpr allows for the targeting of that mechanism for active intervention, and thereby the rational design and selection of anti-HIV compounds.

Rip-1 is the first Vpr associating protein which has been identified in accordance with the present invention, but it is possible that other gene products may either interact with Vpr directly, or indirectly through Rip-1 mediated associations. It has also been discovered in accordance with the present invention, that one or more steroid receptors, especially the glucocorticoid, and GR-type II receptors, may form a multi-part complex with, or are otherwise functionally interactive or combined with, Rip-1 and Vpr, whereby Vpr becomes translocated from the cytoplasm to the nucleus of the human host cell, and there plays an essential role in HIV replication.

U.S. Pat. No. 5,780,220, which is incorporated herein by reference, describes the treatment of individuals exposed to or infected with HIV, by administering to such individuals compounds which are steroid hormone receptor antagonists, particularly glucocorticoid receptor antagonists, and more particularly GR-type II receptor antagonists. Such receptor antagonists inhibit or prevent the replicative and other essential functions of Vpr by interactively blocking the Vpr target in human cells. The use of the glucocorticoid receptor antagonist mifepristone, in the treatment of HIV infected individuals is set forth therein.

There remains a need to identify methods of treating individuals suffering from HIV infection. There remains a need to identify compounds which prevent or inhibit HIV replication in infected cells and thereby are useful for treating individuals suffering from HIV infection. There remains a need to identify methods of treating individuals who have been exposed to HIV to prevent them from becoming HIV infection. There remains a need to identify pharmaceutical compositions useful in such methods.

### SUMMARY OF THE INVENTION

The present invention relates to pharmaceutical composition useful to inhibit HIV replication. The pharmaceutical compositions comprise one or more compounds having a structure selected from the group consisting of mifepristone, the mifepristone metabolite that is hydroxylated, the mifepristone metabolite that is monodemethylated, the mifepristone metabolite that is didemethylated, Compounds D1-D20, and pharmaceutically acceptable salts thereof. The compounds are present in an amount effective to inhibit HIV in an individual. According to the present invention, the composition is a composition formulated as a transdermal patch, a composition formulated as a subdermal delivery system or a controlled/sustained release formulation.

The present invention also relates to methods of treating an individual who is infected with HIV comprising the step of administering to said individual a therapeutically effective amount of a composition that comprises one or more compounds having a structure selected from the group consisting of mifepristone, the mifepristone metabolite that is hydroxylated, the mifepristone metabolite that is monodemethylated, the mifepristone metabolite that is didemethylated, Compounds D1-D20, and pharmaceutically acceptable salts thereof. According to the present invention, the composition is administered as a composition formulated as a transdermal patch, a composition formulated as a subdermal delivery system or a controlled/sustained release formulation.

The present invention further relates to methods of preventing HIV infection in an individual who has been exposed to HIV comprising the step of administering to said individual a prophylactically effective amount of a composition that comprises one or more compounds having a structure selected from the group consisting of mifepristone, the mifepristone metabolite that is hydroxylated, the mifepristone metabolite that is monodemethylated, the mifepristone metabolite that is didemethylated, Compounds D1-D20, and pharmaceutically acceptable salts thereof. According to the present invention, the composition is administered as a composition formulated as a transdermal patch, a composition formulated as a subdermal delivery system or a controlled/sustained release formulation.

The present invention also relates to pharmaceutical compositions that comprise 10-120 mg mifepristone, the mifepristone metabolite that is hydroxylated, the mifepristone metabolite that is monodemethylated, the mifepristone metabolite that is didemethylated, or combinations thereof.

The present invention also relates to methods of treating an individual who is infected with HIV comprising the step of administering to said individual a pharmaceutical compositions that comprise 10-120 mg mifepristone, the mifepristone metabolite that is hydroxylated, the mifepristone metabolite that is monodemethylated, the mifepristone metabolite that is didemethylated, or combinations thereof.

The present invention also relates to methods of preventing HIV infection in an individual who has been exposed to HIV comprising the step of administering to said individual a pharmaceutical compositions that comprise 10-120 mg mifepristone, the mifepristone metabolite that is hydroxylated, the mifepristone metabolite that is monodemethylated, the mifepristone metabolite that is didemethylated, or combinations thereof.

The present invention also relates to methods of treating an individual who is infected with HIV comprising the step of administering to said individual mifepristone, the mifepristone metabolite that is hydroxylated, the mifepristone metabolite that is monodemethylated, the mifepristone metabolite that is didemethylated, or combinations thereof at a dosage level to achieve steady-state serum drug concentration of 17-430 ng/ml.

The present invention also relates to methods of preventing HIV infection in an individual who has been exposed to HIV comprising the step of administering to said individual mifepristone, the mifepristone metabolite that is hydroxylated, the mifepristone metabolite that is monodemethylated, the mifepristone metabolite that is didemethylated, or combinations thereof at a dosage level to achieve steady-state serum drug concentration of 17-430 ng/ml.

The present invention relates to pharmaceutical compositions comprising: a pharmaceutically acceptable carrier or diluent; and, one or more compounds having a structure selected from the group consisting of Formulas D1-D20, the mifepristone metabolite that is hydroxylated, the mifepristone metabolite that is monodemethylated, the mifepristone metabolite that is didemethylated, pharmaceutically acceptable salts thereof and combinations thereof; wherein said compound is present in an amount effective to inhibit HIV in an individual.

### BRIEF DESCRIPTION OF THE FIGURES

FIG. 1 shows the steady-state concentration of mifepristone in the patient serum reported in published studies. In these clinical studies, the steady-state drug concentrations of 35-2300 ng/ml were achieve through daily doses of 1-200 mg.

FIG. 2 shows data related to inhibition of viral replication by mifepristone. U937 cells infected with wild type HIV-1 NL43 virus supplemented with different concentrations of mifepristone. Inhibition of viral replication by mifepristone was dose-dependent.

### DETAILED DESCRIPTION OF PREFERRED EMBODIMENTS

The present invention is useful to therapeutically treat an individual identified as infected with HIV in order to eliminate, reduce or stabilize viral titer and/or increase or stabilize CD4+ cell counts. The present invention is useful to prophylactically treat a high risk individual from becoming infected with HIV.

The compounds of the invention may act as steroid hormone receptor antagonists that interactively blocks Rip-1, alone or in association with one or more steroid receptors, or other components, or one or more steroid receptors alone, preventing or inhibiting formation and translocation of the Vpr/Rip-1 and/or steroid receptor or other component complex.

As used herein, the term “high risk individual” is meant to refer to an individual who is suspected of having been exposed to the HIV virus. Such individuals include health care or other individuals who may have accidentally exchanged blood with an HIV-infected individual, such as through an accidental needle stick, injuries that occur during emergency medical care, rescue or arrest and unprotected sexual contact. High risk individuals can be treated prophylactically before any detection of HIV infection can be made.

As used herein, the term “therapeutically effective amount” is meant to refer to an amount of a compound which produces a medicinal effect observed as reduction or reverse in viral titer and/or and increase or stabilization of CD4+ cell counts when a therapeutically effective amount of a compound is administered to an individual who is infected with HIV. Therapeutically effective amounts are typically determined by the effect they have compared to the effect observed when a composition which includes no active ingredient is administered to a similarly situated individual.

As used herein, the term a “prophylactically effective amount” is meant to refer to an amount of a compound which produces a medicinal effect observed as the prevention of HIV infection in an individual when a prophylactically effective amount of a compound is administered to a high risk individual. Prophylactically effective amounts are typically determined by the effect they have compared to the effect observed when a composition which includes no active ingredient is administered to a similarly situated individual.

The invention provides novel pharmaceutical compositions comprising antiviral compounds that are inhibitors of HIV replication including novel pharmaceutical compositions comprising antiviral compounds provided in specific dosages or in specific drug delivery forms. The antiviral compounds included in the pharmaceutical compositions of the present invention include: mifepristone, which has previously been described as having anti-HIV activity, the mifepristone metabolite that is hydroxylated, the mifepristone metabolite that is monodemethylated, the mifepristone metabolite that is didemethylated, compounds that have a formula selected from the group consisting of Formulas D1-D15, as set forth below, a pharmaceutically acceptable salt thereof or combinations thereof. According to some aspects of the invention, transdermal patches, compositions formulated for subdermal delivery systems and controlled/sustained release formulations are provided which include one or more of: mifepristone, which has previously been described as having anti-HIV activity, the mifepristone metabolite that is hydroxylated, the mifepristone metabolite that is monodemethylated, the mifepristone metabolite that is didemethylated, compounds that have a formula selected from the group consisting of Formulas D1-D15, as set forth below, a pharmaceutically acceptable salt thereof or combinations thereof. According to some aspects of the invention, pharmaceutical compositions are provided which include one or more of mifepristone, the mifepristone metabolite that is hydroxylated, the mifepristone metabolite that is monodemethylated, the mifepristone metabolite that is didemethylated, pharmaceutically acceptable salts thereof or combinations thereof at a dosage of 10-120 mg. According to some aspects of the invention, pharmaceutical compositions are provided which include one or more of mifepristone, the mifepristone metabolite that is hydroxylated, the mifepristone metabolite that is monodemethylated, the mifepristone metabolite that is didemethylated, pharmaceutically acceptable salts thereof or combinations thereof at a dosage adapted to achieve steady-state serum drug concentration of 17-430 ng/ml. According to some aspects of the invention, pharmaceutical compositions are provided which include one or more of the mifepristone metabolite that is hydroxylated, the mifepristone metabolite that is monodemethylated, the mifepristone metabolite that is didemethylated, compounds that have a formula selected from the group consisting of Formulas D1-D15, as set forth below, pharmaceutically acceptable salts thereof or combinations thereof. According to aspects of such compositions are useful to treat individuals who have been infected with HIV as well as to prevent HIV infection in an individual who has been exposed to the virus.

In some embodiments, pharmaceutical compositions of the invention additionally includes one or more additional anti-HIV antiviral compositions such as one or more of mifepristone, zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, interferon alpha-2a, and interferon alpha-2b.

In some embodiments the methods of the invention are used in conjunction with other anti-HIV therapeutic or prophylactic methods. In some embodiments, the methods of the invention further include administration of other antiviral agents such as zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, interferon alpha-2a, and interferon alpha-2b.

Generally, the anti-HIV compounds according to the present invention may be administered by any means that enables the active agent to reach the agent's site of action in the body of the individual. Conventional routes of pharmaceutical administration include parenterally, i.e. intravenous, subcutaneous, intramuscular, orally, transdermally, and subdermally. Pharmaceutical compositions are administered to the individual for a length of time effective to eliminate, reduce or stabilize viral titer and/or increase or stabilize CD4+ cell counts. When used prophylactically, pharmaceutical compositions are administered to the individual for a length of time during which monitoring for evidence of infection continues.

Pharmaceutical compositions of the present invention may be administered either as individual therapeutic agents or in combination with other therapeutic agents. They can be administered alone, but are generally administered with a pharmaceutical carrier selected on the basis of the chosen route of administration and standard pharmaceutical practice.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.001 to 1 grams per kilogram of body weight, in some embodiments about 0.1 to 100 milligrams per kilogram of body weight. Ordinarily dosages are in the range of 0.5 to 50 milligrams per kilogram of body weight, and preferably 1 to 10 milligrams per kilogram per day. In some embodiments, the pharmaceutical compositions are given in divided doses 1 to 6 times a day or in sustained release form is effective to obtain desired results.

Dosage forms (composition) suitable for internal administration generally contain from about 1 milligram to about 500 milligrams of active ingredient per unit. In these pharmaceutical compositions the active ingredient will ordinarily be present in an amount of about 0.5-95 by weight based on the total weight of the composition. Generally, multiple administrations are performed.

According to preferred embodiments of the invention, the antiviral compounds are provided over a course of time in which a therapeutically effective amount of compound is present in the individual's body so as to reduce the viral titer to essentially undetectable levels or essentially undetectable levels such that an asymptomatic individual will not develop symptoms or the onset of such symptoms shall be delayed. According to such preferred embodiments, drug titer remains at antiviral levels in the individual who has been identified as being infected with the virus or who has a high likelihood of having been exposed to the virus for an extended period of time such as 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more days, 48 or more days, 60 or more days or 75 or more days.

Pharmaceutical compositions may be formulated by one having ordinary skill in the art with compositions selected depending upon the chosen mode of administration. Suitable pharmaceutical carriers are described in Remington's Pharmaceutical Sciences, A. Osol, a standard reference text in this field, which is incorporated herein by reference.

For parenteral administration, the compound can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. In some embodiments, a parenteral composition suitable for administration by injection is prepared by dissolving 1.5% by weight of active ingredient in 0.9% sodium chloride solution.

According to some embodiments of the present invention, the composition is administered to tissue of an individual by topically or by lavage. The compounds may be formulated as a cream, ointment, salve, douche, suppository or solution for topical administration or irrigation. Formulations for such routes administration of pharmaceutical compositions are well known. Generally, additives for isotonicity can include sodium chloride, dextrose, mannitol, sorbitol and lactose.

In some cases, isotonic solutions such as phosphate buffered saline are used. Stabilizers include gelatin and albumin. In some embodiments, a vasoconstriction agent is added to the formulation. The pharmaceutical preparations according to the present invention are preferably provided sterile and pyrogen free. The pharmaceutical preparations according to the present invention which are to be used as injectables are provided sterile, pyrogen free and particulate free.

A pharmaceutically acceptable formulation will provide the active ingredient(s) in proper physical form together with such excipients, diluents, stabilizers, preservatives and other ingredients as are appropriate to the nature and composition of the dosage form and the properties of the drug ingredient(s) in the formulation environment and drug delivery system.

In some embodiments, the invention relates to methods of treating patients suffering from HIV infection. In some embodiments, the invention relates to methods of preventing HIV infection in high risk individuals.

According to some embodiments of the invention, the patient is treated with other antiviral therapy in conjunction the administration of pharmaceutical compositions according to the invention. The use of multiple therapeutic approaches provides the patient with a broader based intervention. According to some aspects of the present invention, the individual is also administered another agent. In some embodiments, in combination with administration of the composition, the individual additionally receives compositions that comprises the mifepristone, zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, interferon alpha-2a, and interferon alpha-2b. Other antivirals may also be used delivered according to standard protocols using standard agents, dosages and regimens.

The pharmaceutical compositions according to the present invention may be administered as a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously.

In addition to treating HIV-infected individual, the present invention relates to methods of preventing HIV infection in high risk individuals who, for example, are suspected of having been exposed to the virus.

Additionally, the present invention is particularly useful to prevent recurrence of infection in patients who have been previously diagnosed as HIV positive but show no indication of infection.

Those having ordinary skill in the art can readily identify high risk individuals. Healthcare workers come into contact with infected blood and suffer needle sticks from syringes used on HIV infected individuals. Surgeons cut themselves during surgery. Lab workers, dentists and dental technicians come into contact with infected blood as do emergency medical and rescue workers and law enforcement officers. Individuals involved in athletics and sexually active individuals can also become exposed to the virus. Once any person comes into contact with infected blood, that individual is at an elevated risk of infection.

The present invention is not limited to any particular theory or mechanism of action and while it is currently believed that the compounds identified herein operate through blocking the steroid hormone receptor complex that comprises Rip-1, such explanation of the mechanism of action is not intended to limit the invention. The present invention is further illustrated by the following examples, which are not intended to be limiting in any way.

### EXAMPLES

### Example 1

### Transdermal Drug Delivery

The skin is the largest and most accessible organ of the human body. The permeability of the skin and its ability to deliver drugs to the blood circulation makes it an ideal drug delivery route. Transdermal drug delivery is an increasingly important method of drug administration. Transdermal drug delivery devices typically involve a carrier (such as a liquid, gel, or solid matrix, or a pressure sensitive adhesive) into which the drug to be delivered is incorporated. The drug-containing carrier is then placed on the skin and the drug, along with any adjuvants and excipients, is delivered to the skin.

Typically the portions of the carrier that are not in contact with the skin are covered by a backing. The backing serves to protect the carrier (and the components contained in the carrier, including the drug) from the environment and prevents loss of the ingredients of the drug delivery device to the environment. Backing materials that have found use in transdermal drug delivery devices include metal foils, metalized plastic films, and single layered and multilayered polymeric films.

Transdermal drug delivery utilizes the skin for the delivery of the drug molecules from the surface of the skin, through its layers, to the circulatory system. The transdermal drug delivery technology comprises of a controlling system that regulates the rate of drug delivery to the skin, and another that uses the skin to control the absorption rate.

Transdermal drug delivery occurs in two ways: passive and active transdermal delivery. Passive systems allow the drug to diffuse through the skin into the bloodstream using a simple concentration gradient as a driving force. Active delivery system requires a physical force to facilitate the movement of drug molecules across the skin.

The first transdermal patch was introduced in 1981. Subsequently, the applications of transdermal drug delivery have been expanded to include more products in multiple therapeutic areas. Numerous kinds of medications have been administered through the use of a patch, notably scopolamine for preventing motion sickness, nicotine derivatives intended to discourage an addicted smoker from continuing the smoking habit and estrogen hormones.

Prior art teaches us methods to load and deliver drugs via transdermal routes. U.S. Pat. No. 5,223,261 describes a loading and using a transdermal delivery system for delivering estradiol. U.S. Pat. No. 5,380,760 describes a transdermal delivery system for delivering prostaglandin. U.S. Pat. No. 5,702,720 describes a transdermal delivery system for delivering flurbiprofen. U.S. Pat. No. 6,132,760 describes a transdermal delivery system for delivering testosterone.

The amount of drug that constitutes a therapeutically effective amount varies according to the condition being treated, any drugs being coadministered with the drug, desired duration of treatment, the surface area and location of the skin over which the device is to be placed, and the selection of adjuvant and other components of the transdermal delivery device. Accordingly, it is not practical to enumerate particular preferred amounts but such can be readily determined by those skilled in the art with due consideration of these and other appropriate factors. Generally, however, the drug is present in the adhesive layer in an amount of about 2 to about 9 percent, preferably about 2.5 to about 6.5 percent, by weight based on the total weight of the adhesive layer. A device of the invention preferably contains a therapeutically effective amount of the drug dissolved in the adhesive layer.

The adhesive layer of the device of the invention also comprises one or more polymers, typically one or more copolymers. The polymer(s) utilized in the practice of the invention should be substantially chemically inert to the drug, and is preferably a pressure sensitive skin adhesive. Examples of suitable types of adhesives include acrylates, natural and synthetic rubbers, polysiloxanes, polyurethanes, and other pressure sensitive skin adhesives known in the art, either alone or in combination. Preferably the adhesive is an acrylate copolymer.

### Examples 2

### Delivery of Mifepristone/GR II Antagonists Via Transdermal Patch

One of the issues contributing to the emergence of HIV drug resistance is patient compliance. On the average, HIV+ individuals on Anti Retroviral Therapy (ART) take up to several dozen pills daily. It has been estimated that even as high as 95% compliance in drug regimen could result in >25% eventual drug resistance rate in patients.

The present invention provides transdermal drug delivery devices containing mifepristone, the mifepristone metabolite that is hydroxylated, the mifepristone metabolite that is monodemethylated, the mifepristone metabolite that is didemethylated, Compositions D1-D20 or other GRII antagonists (Drugs). The drug is present in the adhesive layer in a therapeutically effective amount, i.e., an amount effective to allow the device to deliver sufficient amount of the drug to achieve a desired therapeutic result in the treatment of a condition.

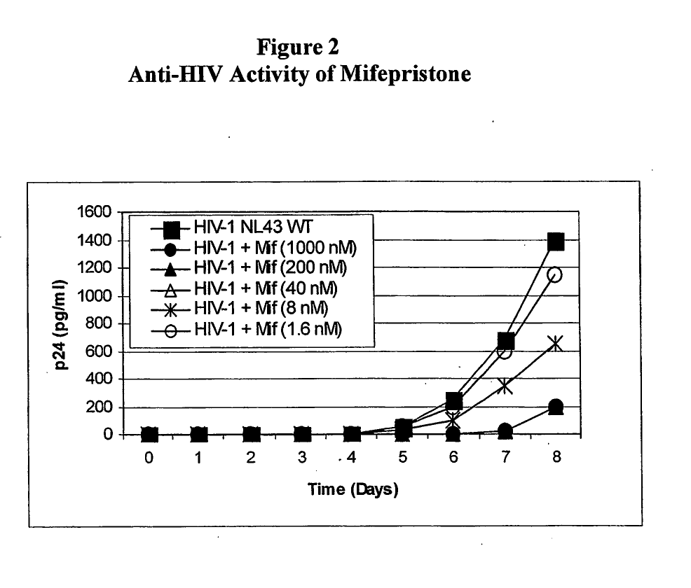

A delivery of mifepristone via a transdermal patch would reduce the number of drugs a patient must take orally and improve compliance. The transdermal drug delivery would be most appropriate in cases where low systemic and steady state drug concentration is desirable. As shown in FIG. 2, mifepristone concentrations of 40-1000 nM (17-430 ng/ml) consistently resulted in inhibition of HIV replication. This delivery method could enhance patient compliance and could reduce the effects of potential drug toxicities.

There are several advantages of delivering anti-viral drugs via transdermal delivery systems. Transdermal drug delivery is not subjected to first-pass effect and does not cause frequent drug concentration alterations as compared to the drugs delivered through the oral route. This reduces the required dose in comparison to the oral drug, delivery. Medications delivered via skin patches avoid liver metabolism and hence allow for lower doses of medication. It also avoids potential toxicity of the drug to the liver. The transdermal drug delivery also offers the flexibility of terminating the drug administration by simply removing the patch from the skin. This delivery system releases a controlled amount of a drug over a long period of time. Transdermal patch systems exhibit slow controlled drug release and absorption and the plasma drug concentration does not vary significantly over time. This delivery method would enhance patient compliance and thereby a reduction of drug resistant viruses as well as reduce the effects of potential drug toxicities.

### Example 3

### Subdermal Drug Delivery (Implantable Devices)

A principal advantage of employing sustained-release compositions is that many therapeutic agents would otherwise be rapidly metabolized or cleared from the patient's system necessitating frequent administration of the drug to maintain a therapeutically effective concentration.

Accordingly, a variety of sustained release devices have been designed for oral, rectal and subcutaneous administration. “Matrix” type devices typically consist of an active compound dispersed in a matrix of carrier material which may be either porous or non-porous, solid or semi-solid, and permeable or impermeable to the active compound. These devices are rather easily prepared; however, they are not suitable for administering some pharmacologically active compounds. In addition, the rate of release of the active compound decreases with time. “Reservoir” type devices consist of a central reservoir of active compound surrounded by a rate controlling membrane (rcm). The rcm is generally a porous or a non-porous material which is non-biodegradable. In the case of the transdermal devices of this type, to maintain an effective concentration of active compound, the rate controlling membrane must have a large surface area. Thus, a common disadvantage of these devices is that their large size makes administration quite inconvenient. Other sustained release devices are hybrid-type devices which contain a matrix core surrounded by a rcm. Yet other devices are mechanical in nature, and include active compound-filled electrical or osmotic pumps.

The subdermally implantable devices of the present invention can be prepared in a variety of sizes and shapes to accommodate such factors as the specific implantation site and the desired release rate of the drug. In a preferred embodiment wherein the drug is a contraceptive agent, the device is substantially cylindrical in shape having a preferred overall length of from about 4.2 cm to about 4.6 cm, and a preferred overall diameter of from about 2.3 mm to about 2.7 mm. In such a case, the central core is rod-shaped, and has a preferred length of from about 3.8 cm to about 4.2 cm, and a preferred diameter of from about 2.0 mm to about 2.2 mm. These dimensions can be modified depending upon such factors as the implantation site and method of implantation, the subject, the condition to be treated, the drug, and the desired release rate of the drug, etc. For example, the length of the implantable device can be varied to deliver different amounts of the drug.

Prior art teaches us methods to load and deliver drugs via subdermal routes. The subdermally implantable devices according to the present invention can be easily fabricated in accordance with standard techniques. Once the drug is mixed with the matrix material to achieve a substantially uniform dispersion, the desired shape of the resultant dispersion is achieved by molding, casting extrusion, or other appropriate process. When the matrix material contains polymers such as silicone elastomers, an additional curing step may be necessary. The intermediate layer is then applied to the thus-shaped matrix, e.g., by swelling, coating or laminating according to known techniques, a polymeric tube in water and then placing it over the matrix and allowing the polymer to dry in place, or by mechanical lapping. The outer layer can likewise be applied in a variety of ways such as by mechanical stretching, swelling or dipping. See, for example, U.S. Pat. Nos. 3,832,252, 3,854,480 and 4,957,119. U.S. Pat. No. 5,756,115 describes a loading and using a subdermal delivery system for delivering contraceptives. The dimensions of the implant are also determined on the basis of the implantation method. The devices of the present invention can be implanted into a subject in accordance with standard procedures.

The present invention provides subdermal drug delivery devices containing mifepristone, the mifepristone metabolite that is hydroxylated, the mifepristone metabolite that is monodemethylated, the mifepristone metabolite that is didemethylated, Compositions D1-D5 or other GRII antagonists (Drugs). The drug is present in the implantable devices in a therapeutically effective amount, i.e., an amount effective to allow the device to deliver sufficient amount of the drug to achieve a desired therapeutic result in the treatment of a condition.

### Example 4

### Sustained and Controlled Release Drug Delivery

To improve the effectiveness of drug therapy and to reduce possible systematic side effects, many attempts have been made to deliver drugs in a controlled profile to human patients. The advantages of controlled release dosage forms are well known in both the pharmaceutical and medical sciences. The therapeutic benefits of controlled-release dosage forms include the pharmacokinetic ability to maintain a preplanned blood level of an administered drug over a comparatively longer period of time. The therapeutic benefits include also a simultaneous increase in patient compliance and a reduction in the number of doses of drug administered to a patient.

The prior art made available controlled release dosage that sought to provide a drug release rate profile that matched the blood physiological and chrono-pharmacological requirements needed for therapy. For example, an osmotic dosage form for delivering various drugs to a patient environment of use is presented in U.S. Pat. No. 3,845,770 issued to patentees Theeuwes and Higuchi, and in U.S. Pat. No. 3,916,899 issued to the same patentees. The dosage forms disclosed in these patents are manufactured comprising a wall that surrounds a compartment comprising a drug with an exit in the wall for delivering the drug to a patient. In U.S. Pat. Nos. 4,008,719; 4,014,334; 4,058,122; 4,116,241; and 4,160,452 patentees Theeuwes and Ayer made available dosage forms comprising an inside and an outside wall made of poly(cellulose acylate) for delivering a dosage of drug to a patient in need thereof.

Additional semipermeable polymers comprise acetaldehyde dimethylcellulose acetate; cellulose acetate ethylcarbamate; cellulose acetate methylcarbamate; cellulose diacetate propylcarbamate; cellulose acetate diethylaminoacetate; ethyl acrylate methyl methacrylate, semipermeable polyamide; semipermeable polyurethane; semipermeable sulfonated polystyrene; semipermeable crosslinked selective polymer formed by the coprecipation of a polyanion and polycation, as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable, lightly crosslinked polystyrenes; semipermeable crosslinked poly(sodium styrene sulfonate); semipermeable crosslinked poly (vinylbenzyltrimethyl ammonium chloride); and semipermeable polymers possessing a fluid permeability in the range of 2.5×10⁻⁸ to 5×10⁻² (cm²/hr multidot atm), expressed per atmosphere of hydrostatic or osmotic pressure difference across the semipermeable exterior wall 12. The polymers are known to the polymer art in U.S. Pat. Nos. 3,845,770; 3,916,899 and 4,160,020; and in Handbook of Common Polymers, by Scott, J. R. and Roff, W. J. 1971, CRC Press, Cleveland, Ohio. Wall 12, in a present manufacture can be coated from a substantially single solvent system, such as acetone if coated from a solution, or water if coated as a dispersion.

The present invention provides delivery of mifepristone, the mifepristone metabolite that is hydroxylated, the mifepristone metabolite that is monodemethylated, the mifepristone metabolite that is didemethylated, Compositions D1-D20 or other GRII antagonists (Drugs) via a sustained release or controlled release delivery techniques.

### Example 5

### Effective Clinical Dosage for Mifepristone

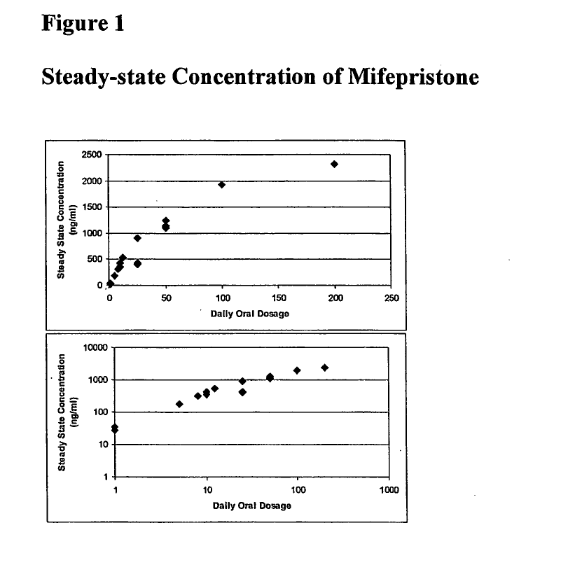

Mifepristone [11β-(4dimethylaminophenyl)-17β-hydroxy-17-α-(propyl-lynyl)-4,9-dien-3-one] is a glucocorticoid receptor antagonist with a molecular weight of 429.6 (C29H35NO2). Several studies have reported on the daily oral administration of mifepristone (multiple dosing) [1-7]. The steady-state concentrations of mifepristone in the patient serum reported in these studies are compiled in FIG. 1. In these clinical studies, the steady-state drug concentrations of 35-2300 ng/ml were achieve through daily doses of 1-200 mg (4-30 days).

Mifepristone has been shown to be effective in inhibiting HIV replication in vitro doses ranging from 40 nM to 1000 nM, and the IC50 of mifepristone was determined to be 8 nM (FIG. 2). In several laboratory adapted and clinical isolate viruses, mifepristone concentrations of 40-1000 nM (17-430 ng/ml) consistently resulted in >90% inhibition of viral replication (Table 1).

These results indicate that the anti-viral activities of mifepristone observed between the concentrations of 17-430 ng/ml in cell culture tests can be achieved in humans by a daily mifepristone administration of 1-100 mg.

TABLE 1
Summary of Mifepristone's Anti-viral Effects In Vitro

| | ARRRP | Lab/Clinical | Coreceptor | Maximum % Reduction in Viral Replication |
|---|---|---|---|---|
| Virus/Cells | Catalog # | Isolate | Usage | By Mifepristone in PBMCs |
| HIV-1 BaL | 510 | Lab | R5 (NSI) | >90% |
| HIV-1 89.6 | 1966 | Lab | R5X4 | >90% |
| HIV-1 Ada | 416 | Lab | R5 (NSI) | >90% |
| HIV-1 NL43 | 114 | Lab | R5 (NSI) | >90% |
| 91US054 | 2101 | Clinical | R5 (NSI) | >95% |
| 91US056 | 2099 | Clinical | R5 (NSI) | >95% |
| 92US657 | 2053 | Clinical | R5 (NSI) | >80% |
| 92US660 | 1722 | Clinical | R5 (NSI) | >90% |
| 92US714 | 2055 | Clinical | R5 (NSI) | >90% |
| 92US723 | 2056 | Clinical | R5X4 | >90% |
| 92US727 | 2057 | Clinical | R5 (NSI) | >95% |
| U1 | 165 | Infected Cell | | >95% |
| J1.1 | 1340 | Infected Cell | | >95% |
| LL58 | 811 | Infected Cell | | >90% |
| ACH-2 | 349 | Infected Cell | | >95% |
| OM-10.1 | 1319 | Infected Cell | | >95% |
| HIV+ | N/A | Infected PBMCs | | >95% |
| Patient 1 | | | | |
| HIV+ | N/A | Infected PBMCs | | >95% |
| Patient 2 | | | | |
| HIV+ | N/A | Infected PBMCs | | >90% |
| Patient 3 | | | | |
| HIV+ | N/A | Infected PBMCs | | >95% |
| Patient 4 | | | | |

### Example 4

### Mifepristone Metabolites

Unbound Mifepristone is metabolized by two-step demethylation or by hydroxylation, and the initial metabolic steps are catalysed by the cytochrome P450 (CYP) enzyme CYP3A4 (Jang et al., 1996 Biochem. Pharmacol. 52:753-761 and Reilly et al, 1999, which are incorporated herein by reference). Three metabolites of Mifepristone have been identified (Sarkar, 2002 Eur. J. of Obstetrics & Gynecol and Reprod. Biol. 101:113-120). This compound undergoes demethylation to produce monodemethylated and di-demethylated derivatives as well as hydroxylation of the propynyl group to yield hydroxylated metabolite. Studies have shown that the metabolism of Mifepristone to mono-demethylated and hydroxylated metabolites was rapid but removal of the second methyl group leading to the formation of di-demethylated derivative occurred much more slowly and to much lesser extent than removal of the first. Serum levels of the monodemethylated metabolite always exceeded those of Mifepristone (Sarkar, 2002). The concentrations of the didemethylated and hydroxylated metabolites equalled or exceeded those of Mifepristone when the ingested dose was 400 mg or more. Monodemethylation and hydroxylation were rapid high-capacity reactions, whereas didemethylation was a lower-capacity reaction (Sarkar, 2002).

In each group of different dosage, positive correlations were found between the individual mean alpha 1-acid glycoprotein (AAG) concentrations and the peak concentration of Mifepristone measured at 1-2 h, versus the plateau concentration of Mifepristone measured at 6 h. The in-vitro studies showed that AAG was saturated by Mifepristone concentrations exceeding 2.5 microM. In serum at 40 nM and 2.5 microM Mifepristone concentrations, 2.7% and 2.4%, respectively, of Mifepristone was not protein bound. These results suggest that AAG regulates in part the serum concentrations of Mifepristone, and Mifepristone exceeding the specific serum transport capacity is effectively metabolized.

Like Mifepristone, these metabolites are immunologically and biologically active and retain anti-progestational and anti-glucocorticoid properties. The relative binding affinities of the metabolites to the human glucocorticoid receptor are 61, 48 and 45% for the monodemethylated, hydroxylated, and didemethylated metabolites, respectively; each was higher than that of dexamethasone or cortisol (23%).

The data on Table 2 show comparative anti-progesterone, and anti-glucocorticoid activities and the comparative ration of such activities for the mifepristone metabolites D6, D7 and D8. Mifepristone derivatives exhibiting reduced abortefaceint activity provide an advantage over mifepristone with respect to safety and elimination of side effects.

TABLE 2

| | Anti- | Anti- | |
|---|---|---|---|
| | Progesterone | Glucocorticoid | A-G/A-P |
| mono-demethylated | 21% | 61% | 290% |
| mifepristone metabolite D6 | | | |
| di-demethylated | 9% | 45% | 500% |
| mifepristone metabolite D7 | | | |
| hydroxylated | 15% | 48% | 320% |
| mifepristone metabolite D8 | | | |

Structures

Mifepristone has the following structure:

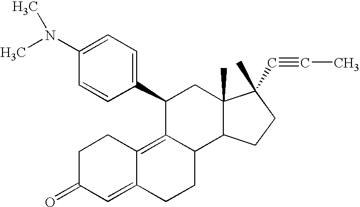

The mono-demethylated mifepristone metabolite has the following structure:

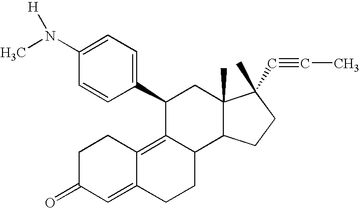

The di-demethylated mifepristone metabolite has the following structure:

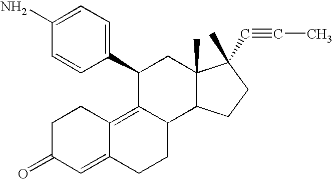

The hydroxylated mifepristone metabolite has the following structure:

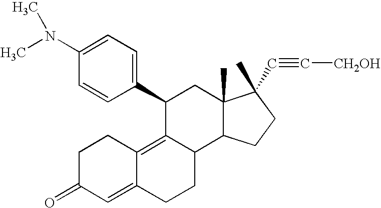

D1, Pregna-4,6-diene-3,20-dione, has the following structure: 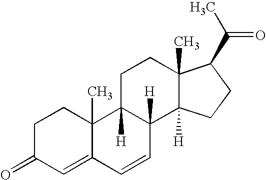
D1 is available as Sigma Product Number: R19,725-4 and MDL Number: MFCD00199858, and was described in GB 929271 and U.S. Pat. No. 3,362,968, which are each incorporated herein by reference. In some embodiments, other compounds described in GB 929271 and U.S. Pat. No. 3,362,968 may be employed according to the present invention.

D2, 17-α-ethynyl-17-β-hydroxyestr-5 (10)-En-3-one, has the following structure: 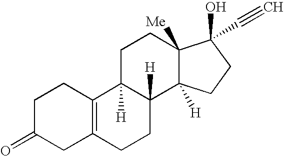
D2 is available as Sigma Product Number: R18,844-1 and MDL Number: MFCD00199015 and is described in U.S. Pat. No. 3,024,256, which is incorporated herein by reference. In some embodiments, other compounds described in U.S. Pat. No. 3,024,256 may be employed according to the present invention

D3, Epoxyazadiradione, has the following structure: 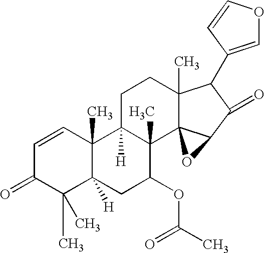
D3 is described in Indian patents, IN 33649 and IN 67932 and PCT publication WO92/19616, which are each incorporated herein by reference. In some embodiments, other compounds described in IN 33649 and IN 67932 and PCT publication WO92/19616, may be employed according to the present invention

D4, NSC641295, Astragaloside II, has the following structure: 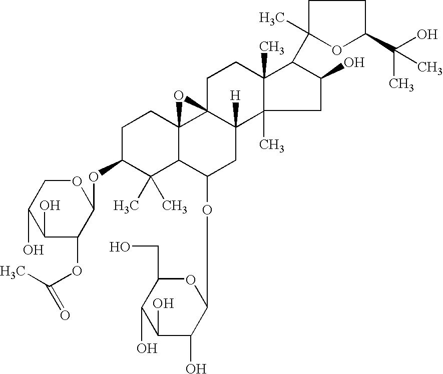
D4 is described in JP 62012791 and WO01/01996, which are each incorporated herein by reference. In some embodiments, other compounds described in JP 62012791 and WO01/01996 may be employed according to the present invention

D5, 3′Azido-3′deoxy-5′-O-[(11-.beta.-hydroxy-3-oxo-17-.beta.-androst-4-enyl)carbonyl]thymidine (Combination of Hydrocortisone Acetate and Zidovudine), has the following structure: 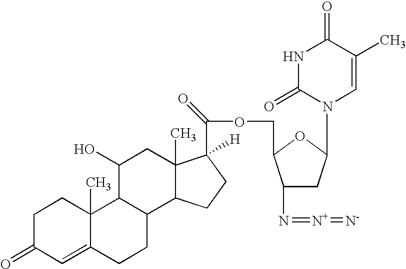
D5 is a combination of Hydrocortisone Acetate and Zidovudine. Hydrocortisone Acetate is available as Sigma Product Number: H4126; Zidovudine is available as Sigma Product Number: 11546.

Compound D6 refers to pregnenolone, 16-alpha-carbonitrile which is disclosed in Cell 1998, 92:73 and US Application Publication No. 2002/0085995, which are each incorporated herein by reference. In some embodiments, other compounds described in US Application Publication No. 2002/0085995 may be employed according to the present invention.

Compound D7 refers to promegestrone, which is disclosed in U.S. Pat. No. 4,911,916, which is incorporated herein by reference. In some embodiments, other compounds described in U.S. Pat. No. 4,911,916, may be employed according to the present invention.

Compound D8 refers to progesterone which is disclosed in J. Steroid. Biochem. 1988, 29:600, Endocrinol. 1980, 107:118 and U.S. Pat. No. 2,142,170, which are each incorporated herein by reference. In some embodiments, other compounds described in U.S. Pat. No. 2,142,170, may be employed according to the present invention.

Compound D9 refers to cortexolone which is disclosed in Endocrinology 1980, 107: 117 and U.S. Pat. No. 3,651,049, which are each incorporated herein by reference. In some embodiments, other compounds described in U.S. Pat. No. 3,651,049, may be employed according to the present invention.

Compound D10 refers to 6-beta-bromogesterone which is disclosed in Endocrinology 1980, 107: 119, which is incorporated herein by reference.

Compound D11 refers to RU43044 which is disclosed in PNAS 1992, 89:3571 and U.S. Application Publication No. 2002/0169152, which are each incorporated herein by reference. In some embodiments, other compounds described in U.S. Application Publication No. 2002/0169152, may be employed according to the present invention.

Compound D12 refers to RU40555 which is disclosed in J. Endcrinol. 2001, 169:309 and PCT Published Application No. WO00/21509, which are each incorporated herein by reference. In some embodiments, other compounds described in PCT Published Application No. WO00/21509, may be employed according to the present invention.

Compound D13 refers to spironolactone which is disclosed in Laryngoscope 2002, 112: 298 and U.S. Pat. No. 3,143,288, which are each incorporated herein by reference. In some embodiments, other compounds described in U.S. Pat. No. 3,143,288, may be employed according to the present invention.

Compound D14 refers to onapristone which is disclosed in Biol Pharm. Bull 2002, 25: 1223, J. Biolog. Chem. 2000, 275: 17771 and U.S. Pat. No. 5,719,136, which are each incorporated herein by reference. In some embodiments, other compounds described in U.S. Pat. No. 5,719,136, may be employed according to the present invention.

Compound D15 refers to cyproterone acetate which is disclosed in Mol. Pharm 2003, 63:1012 and U.S. Application Publication No. 2004/0087563, which are each incorporated herein by reference. In some embodiments, other compounds described in U.S. Application Publication No. 2004/0087563, may be employed according to the present invention.

Compound D16 refers to trans 4-hydroxytamoxifen which is disclosed in J. Biolog. Chem. 2000, 275: 17771 and U.S. Pat. No. 4,973,755, which are each incorporated herein by reference. In some embodiments, other compounds described in U.S. Pat. No. 4,973,755, may be employed according to the present invention.

Compound D17 refers to RTI-3021-012 which is disclosed in Endocrinology 1999, 140:1449, which is incorporated herein by reference.

Compound D18 refers to RTI-3021-022 which is disclosed in Endocrinology 1999, 140:1450, which is incorporated herein by reference.

Compound D19 refers to actinomycin D which is disclosed in J. Pharmacol. Exp. Ther. 1980, 212: 225 and U.S. Pat. No. 3,954,970, which are each disclosed herein by reference. In some embodiments, other compounds described in U.S. Pat. No. 3,954,970, may be employed according to the present invention.

Compound D20 refers to cycloheximide which is disclosed in J. Pharmacol. Exp. Ther. 1980, 212: 226 and U.S. Pat. No. 3,214,431, which are each incorporated herein by reference. In some embodiments, other compounds described in U.S. Pat. No. 3,214,431, may be employed according to the present invention.

### REFERENCES

References cited herein including those set forth below are hereby incorporated herein by reference.
1. M. L. Swahn, G. Wang, A. R. Aedo, S. Z. Cekan and M. Bygdeman, Plasma levels of anti progestin RU486 following oral administration to non-pregnant and early pregnant women. Contraception 34 (1986), pp. 469-481.
2. O. Heikinheimo, Kontula K, H. Croxatto Spitz I, Luukkainen T, Lahteenmaki P Pharmacokinetics of the antiprogestin RU486 in women during multiple doses administration. J. Steroid Biochem 32 (1989), pp. 21-25.
3. I. Foldesi, G. Falkay and L. Kovacs, Determination of RU486 (mifepristone) in blood by radioreceptor assay: a pharmacokinetic study. Contraception 56 (1996), pp. 27-32.
4. R. Kekkonen, O. Heikinheimo, E. Mandelin and P. Lahteenmaki, Pharmacokinetics of mifepristone after low oral doses. Contraception 54 (1996), pp. 229-234.
5. N. N. Sarkar. Mifepristone: bioavailability, pharmacokinetics and use-effectiveness. 2002. Euro J Obst Gyneco and Repro Bio. 101:113-120.
6. Croxatto, H B, Salvatierra A M, Croxatto, H D, Fuentealba B. Effects of continuous treatment with low dose mifepristone throughout one menstrual cycle. Human Reprod. 1992. 7:945-50.
7. O. Heikinheimo, Kekkone R. Dose-response relationships of RU486. Ann Med. 1993.25: 71-6.

### CROSS REFERENCE TO RELATED APPLICATIONS

This application claims the benefit of U.S. provisional Ser. No. 60/480,500, filed Jun. 20, 2003 and U.S. provisional Ser. No. 60/480,393, filed Jun. 20, 2003. The entire disclosures of each of these applications are incorporated herein by reference.

1. A pharmaceutical composition comprising:
a pharmaceutically acceptable carrier or diluent; and,
one or more compounds having a structure selected from the group consisting of mifepristone, Formulas D1-D20, and pharmaceutically acceptable salts thereof;
wherein said compound is present in an amount effective to inhibit HIV in an individual, said composition being in the form selected from the group consisting of:
a composition formulated as a transdermal patch;
a composition formulated as a subdermal delivery system; and
a controlled/sustained release formulation.

2. The pharmaceutical composition of claim 1 further comprising one or more compounds having a structure selected from the group consisting: zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscamet, interferon alpha-2a, and interferon alpha-2b.

3-6. (canceled)

7. The pharmaceutical composition of claim 1 wherein said composition comprises 10-120 mg mifepristone.

8. The pharmaceutical composition of claim 1 wherein said composition comprises 60 mg mifepristone.

9. The pharmaceutical composition of claim 1 wherein said composition comprises 30 mg mifepristone.

10. A method of treating an individual who is infected with HIV comprising the step of administering to said individual a therapeutically effective amount of a composition according to claim 1.

11. The method of claim 10 wherein said individual is administered mifepristone at a dosage level to achieve steady-state serum drug concentration of 17-430 ng/ml.

12. A method of preventing HIV infection in an individual identified as being a high risk individual, the method comprising the step of administering to said individual a prophylactically effective amount of a composition according to claim 1.

13. A method of claim 12 wherein said individual is administered mifepristone at a dosage level to achieve steady-state serum drug concentration of 17-430 ng/ml.

14-20. (canceled)

21. A pharmaceutical composition comprising: a pharmaceutically acceptable carrier or diluent; and, one or more compounds having a structure selected from the group consisting of: hydroxylated mifepristone metabolite, monodemethylated mifepristone metabolite, didemethylated mifepristone metabolite, Compounds D1-D20, and pharmaceutically acceptable salts thereof; wherein said compound is present in an amount effective to inhibit HIV in an individual.

22. The pharmaceutical composition of claim 21 further comprising one or more compounds having a structure selected from the group consisting: mifepristone, zidovudine (AZT), abacavir, 3TC, d4T, ddl, ddC, efavirenz, nevirapine, delavidine, amprenavir, Indinavir, Lopinavir, nelfinavir, ritonavir, sanquinavir, acyclovir, ganciclovir, foscarnet, interferon alpha-2a, and interferon alpha-2b

23. The pharmaceutical composition of claim 21 comprising 10-120 mg hydroxylated mifepristone metabolite, monodemethylated mifepristone metabolite, didemethylated mifepristone metabolite, a pharmaceutically acceptable salt thereof or a combination thereof.

24. The pharmaceutical composition of claim 21 wherein said composition comprises 60 mg hydroxylated mifepristone metabolite, monodemethylated mifepristone metabolite, didemethylated mifepristone metabolite a pharmaceutically acceptable salt thereof or a combination thereof.

25. The pharmaceutical composition of claim 21 wherein said composition comprises 30 mg hydroxylated mifepristone metabolite, monodemethylated mifepristone metabolite, didemethylated mifepristone metabolite a pharmaceutically acceptable salt thereof or a combination thereof.

26. A method of treating an individual who is infected with HIV comprising the step of administering to said individual a therapeutically effective amount of a composition according to claim 21.

27. A method of preventing HIV infection in an individual identified as being a high risk individual, the method comprising the step of administering to said individual a prophylactically effective amount of a composition according to claim 21.
